SEQUENCE LISTING (24 sequences)

SEQ ID NO: 1 - DNA, 48 bp, Synthetic Construct
  source 1..48: mol_type = other DNA
ctcgggacgacgccagaacgtcaggaggatatggtaacatagtcgtcc

SEQ ID NO: 2 - DNA, 48 bp, Synthetic Construct
  source 1..48: mol_type = other DNA
ctcgggacgacgccagaaagatatgaggatagggcgacctagtcgtcc

SEQ ID NO: 3 - DNA, 48 bp, Synthetic Construct
  source 1..48: mol_type = other DNA
ctcgggacgacgccagaaagatatgaggataggtacgcctagtcgtcc

SEQ ID NO: 4 - DNA, 48 bp, Synthetic Construct
  source 1..48: mol_type = other DNA
ctcgggacgacgccagaaaggtatgaggataggctcgcctagtcgtcc

SEQ ID NO: 5 - DNA, 48 bp, Synthetic Construct
  source 1..48: mol_type = other DNA
ctcgggacgacgccagaaaggtatgaggatagggctccctagtcgtcc

SEQ ID NO: 6 - DNA, 48 bp, Synthetic Construct
  source 1..48: mol_type = other DNA
ctcgggacgacgccagaacgataggaggatatgcctgcatagtcgtcc

SEQ ID NO: 7 - DNA, 48 bp, Synthetic Construct
  source 1..48: mol_type = other DNA
ctcgggacgacgccagaacgataggaggataggcttgcctagtcgtcc

SEQ ID NO: 8 - DNA, 48 bp, Synthetic Construct
  source 1..48: mol_type = other DNA
ctcgggacgacgccagaacgataggaggatatgcttgcatagtcgtcc

SEQ ID NO: 9 - DNA, 48 bp, Synthetic Construct
  source 1..48: mol_type = other DNA
ctcgggacgacgccagaacgataggaggatagggctacctagtcgtcc

SEQ ID NO: 10 - DNA, 48 bp, Synthetic Construct
  source 1..48: mol_type = other DNA
ctcgggacgacgccagaacgataggaggatagggctccctagtcgtcc

SEQ ID NO: 11 - DNA, 48 bp, Synthetic Construct
  source 1..48: mol_type = other DNA
ctcgggacgacgccagaacgataggaggataggtttacctagtcgtcc

SEQ ID NO: 12 - DNA, 48 bp, Synthetic Construct
  source 1..48: mol_type = other DNA
ctcgggacgacgccagaacgtaaggaggataggcagacctagtcgtcc

SEQ ID NO: 13 - DNA, 48 bp, Synthetic Construct
  source 1..48: mol_type = other DNA
ctcgggacgacgccagaacgtaaggaggatatgcctgcatagtcgtcc

SEQ ID NO: 14 - DNA, 48 bp, Synthetic Construct
  source 1..48: mol_type = other DNA
ctcgggacgacgccagaacgtaaggaggataggctcgcctagtcgtcc

SEQ ID NO: 15 - DNA, 48 bp, Synthetic Construct
  source 1..48: mol_type = other DNA
ctcgggacgacgccagaacgtaaggaggatatgctcgcatagtcgtcc

SEQ ID NO: 16 - DNA, 48 bp, Synthetic Construct
  source 1..48: mol_type = other DNA
ctcgggacgacgccagaacgtaaggaggataggcttgcctagtcgtcc

SEQ ID NO: 17 - DNA, 48 bp, Synthetic Construct
  source 1..48: mol_type = other DNA
ctcgggacgacgccagaacgtaaggaggatatgcttgcatagtcgtcc

SEQ ID NO: 18 - DNA, 48 bp, Synthetic Construct
  source 1..48: mol_type = other DNA
ctcgggacgacgccagaacgtaaggaggatatggttacatagtcgtcc

SEQ ID NO: 19 - DNA, 48 bp, Synthetic Construct
  source 1..48: mol_type = other DNA
ctcgggacgacgccagaacgtaaggaggataggtttacctagtcgtcc

SEQ ID NO: 20 - DNA, 48 bp, Synthetic Construct
  source 1..48: mol_type = other DNA
ctcgggacgacgccagaacgttaggaggataggcttgcctagtcgtcc

SEQ ID NO: 21 - DNA, 48 bp, Synthetic Construct
  source 1..48: mol_type = other DNA
ctcgggacgacgccagaacgttaggaggatatgcttgcatagtcgtcc

SEQ ID NO: 22 - DNA, 48 bp, Synthetic Construct
  source 1..48: mol_type = other DNA
ctcgggacgacgccagaacgttaggaggatagggcaacctagtcgtcc

SEQ ID NO: 23 - DNA, 48 bp, Synthetic Construct
  source 1..48: mol_type = other DNA
ctcgggacgacgccagaacgttaggaggatagggctccctagtcgtcc

SEQ ID NO: 24 - DNA, 48 bp, Synthetic Construct
  source 1..48: mol_type = other DNA
ctcgggacgacgccagaacgttaggaggataggtttacctagtcgtcc

(19) United States
(12) Patent Application Publication
(10) Pub. No.: US 2025/0147052 A1
(43) Pub. Date: May 8, 2025

(54) Aptamers for Cortisol and Other Hormone Sensing, Aptamer-Based Sensors, and a Method for Optimizing Conformation-Switching Aptamers for Biosensing Applications

(71) Applicant: Government of the United States, as represented by the Secretary of the Air Force, Wright-Patterson AFB, OH (US)

(72) Inventors: Monica Wolfe, Dorchester, MA (US); Jorge Chavez Benavides, Kettering, OH (US); Alyssa Cramer, Dayton, OH (US); Sean Webb, Dayton, OH (US); Yaroslav Chushak, Beavercreek, OH (US); Peter Mirau, Beavercreek, OH (US)

(21) Appl. No.: 18/925,182

(22) Filed: Oct. 24, 2024

Related U.S. Application Data
(60) Provisional application No. 63/547,442, filed on Nov. 6, 2023.

(51) Int. Cl. G01N 33/74; C12N 15/115; G01N 33/53

(52) U.S. Cl. CPC G01N 33/743; C12N 15/115; G01N 33/5308; C12N 2310/16; C12N 2310/531

(57) ABSTRACT

Aptamers, biosensors and aptamer-based biosensors for monitoring cortisol and other hormones are provided. Methods of optimizing aptamers for biosensing applications are also provided. Aptamers have been optimized to undergo a conformational change when binding, or unbinding, cortisol at physiologically relevant concentrations. These aptamers can be incorporated into aptamer-based sensors for real-time, continuous monitoring of cortisol.

### CROSS-REFERENCE TO A RELATED APPLICATION

Pursuant to 37 C.F.R. § 1.78 (a) (4), this application claims the benefit of and priority to prior filed co-pending Provisional Application Ser. No. 63/547,442, filed Nov. 6, 2023, which is expressly incorporated herein by reference.

### RIGHTS OF THE GOVERNMENT

The invention described herein may be manufactured and used by or for the Government of the United States for all governmental purposes without the payment of any royalty.

### SEQUENCE LISTING

The ASCII Sequence Text File named AFD-2438.xml that was created on Sep. 16, 2024, and has a size 23,000 bytes is hereby incorporated by reference in the present application its entirety.

### FIELD OF THE INVENTION

The present invention relates generally to aptamers, biosensors and, more particularly, to aptamer-based biosensors for monitoring cortisol and other hormones. Methods of optimizing aptamers for biosensing applications are also provided.

### BACKGROUND OF THE INVENTION

Stress and fatigue monitoring is of great interest for human health and performance. Stress hormones, such as cortisol, a small molecule steroid hormone, play an important role in the stress-responsive hypothalamus-pituitary-adrenal (HPA) axis and are involved in a range of bodily processes, including proteolysis, glucose regulation, blood pressure, and immune responses. Cortisol dysregulation occurs in a number of conditions and has been linked to fatigue, obesity, post-traumatic stress disorder, anxiety disorders, depression, and overall performance.

Real-time, continuous, and non-invasive monitoring of cortisol levels in the body would play an important role in the prevention of negative performance outcomes and the diagnosis and management of disease. Cortisol is present at nanomolar concentrations in blood and recent clinical studies have shown a correlation between cortisol levels in blood and other bodily fluids such as sweat, saliva, urine, and interstitial fluid (ISF). ISF, in particular, is attractive for biosensing because it has a composition similar to blood plasma but, unlike blood, can be drawn continuously from the dermis using noninvasive and compliance-free methods.

Electrochemical, aptamer-based (EAB) sensors have the capability to support continuous, real-time monitoring of specific target molecules in complex biofluids. Recently, EAB sensing technology was incorporated onto microneedle sensor arrays for continuous sensing of molecules in ISF in vivo.

Structure-switching aptamers (SSA) are utilized as the bio-recognition element in EAB sensing platforms due to their ability to undergo a conformational change in the presence of target molecule, thereby moving a redox reporter close to or away from the surface of an electrode. While the field of SSAs has grown significantly over the past decade, the process of converting a traditional aptamer into a SSA remains challenging and relies largely on trial and error.

Structure-switching or capture Systematic Evolution of Ligands by Exponential Enrichment (SELEX) is a newer technique that relies on the presence of a complementary DNA strand that forms a duplex with the aptamer sequence. Upon binding, the aptamer undergoes a conformational change and the strand is displaced by the target molecule. The SELEX protocol and aptamers are described in U.S. Pat. No. 5,270,163 entitled “Methods for Identifying Nucleic Acid Ligands” in the name of Gold, et al., which is incorporated into the Detailed Description herein by reference. Recently, capture SELEX was used to identify a group of cortisol binding aptamers. Practically, however, these aptamers do not undergo sufficient structural changes in the absence of the capture strand and required further optimization in order to maximize signal output for EAB sensors. Additionally, the sensitivity of these aptamers in biofluids is insufficient for detecting cortisol at physiological concentrations.

Therefore, a need exists for improved cortisol-binding aptamers that undergo a conformational change when binding, or unbinding, cortisol at physiologically relevant concentrations. A need also exists for such aptamers that can be incorporated into aptamer-based sensors for real-time, continuous monitoring of cortisol.

### SUMMARY OF THE INVENTION

The present invention relates generally to aptamers, biosensors and, more particularly, to aptamer-based biosensors for monitoring cortisol and other hormones. Methods of optimizing aptamers for biosensing applications are also provided.

While the invention will be described in connection with certain embodiments, it will be understood that the invention is not limited to these embodiments. To the contrary, this invention includes all alternatives, modifications, and equivalents as may be included within the spirit and scope of the present invention.

In some embodiments, aptamers are provided. The aptamers may comprise any one of SEQ IDs 1-24. In some cases, the aptamers may be selected from the group consisting of SEQ IDs 1-24. The sequences may also include any modifications, variants, concatenations, and/or truncations thereto, and in general may include any sequence with substantial or significant homology or sequence identity with the aptamer sequences described herein. The aptamers may be short single-stranded DNA sequences that have been optimized to bind cortisol. These sequences may be variations of existing aptamers that are truncated and mutated to improve cortisol binding affinity and structure switching capability in relevant biofluids. In some embodiments, the aptamers may, therefore, be structure-switching aptamers (SSAs).

In some embodiments, aptamer-based biosensors are provided. The biosensors can be of any suitable type including, but not limited to: electrochemical, colorimetric, or fluorescence based sensors. In some cases, the sequences can be functionalized with a redox reporter (e.g., methylene blue (MB)) and immobilized onto a sensor surface for electrochemical detection of cortisol in biofluids.

In some embodiments, methods of cortisol and other hormone detection are provided. The methods may comprise the steps of contacting a sample with the aptamer-based biosensors described herein, and detecting cortisol or another hormone in the sample. The detection may comprise measuring a signal generated upon binding of the cortisol or other hormone to the aptamer in the aptamer-based biosensor.

In some embodiments, methods of optimizing aptamers for biosensing applications are provided. In some cases, the aptamers may be conformation-switching. In some cases, a method of optimizing aptamers for biosensing applications may comprise the steps of:
a) structurally characterizing an aptamer at the molecular level using nuclear magnetic resonance (NMR) and computer modeling to identify sequence-specific binding features and conformations, wherein said structural characterization is performed:
   1) in the absence of a target molecule; and
   2) in the presence of a target molecule;
b) performing large-scale mutational analysis using a microarray-based platform to allow for simultaneous assessment of a plurality of aptamer point mutations and screening of combinatorial mutations for improved binding affinity; and
c) characterizing aptamer binding affinity wherein binding affinity is evaluated in at least one of: 1) buffer, 2) biofluid.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flow chart showing of one embodiment of the bio-recognition element (BRE) discovery and optimization pipeline.

FIG. 1A is a diagram of the MFold-predicted secondary structure of CSS.3 showing location of cut sites for Stem #1 truncation.

FIG. 1B is a graph that shows NMR spectra of aptamer variants in the absence (black) or presence (gray) of cortisol showing loss of structure with stem truncation.

FIG. 1C is a graph that shows the 800 MHZ 2D NOESY NMR spectra of CSS.3_cut3 in the presence of cortisol.

FIG. 1D is a graph that shows a cross section through the 2D NOESY spectrum showing magnetization exchange from the cortisol methyl protons both show the interaction of the GA base pair with cortisol.

FIG. 1E shows a rigid 3D structure of CSS.3_cut3 in its binding conformation with predicted non-canonical GA base pair highlighted in green.

FIG. 1F is 3D model of CSS.3_cut3 aptamer docked to cortisol with binding pocket highlighted in magenta.

FIG. 2A is a table showing calculated binding affinities from steady state equilibrium to cortisol in sISF.

FIG. 2B is a graph of the replicate traces showing real-time baseline, association and dissociation.

FIG. 2C is a graph where the averaged steady state binding traces for stem-optimized CSS.3_cut3 aptamer are featured (where SD is standard deviation).

FIG. 3A is a schematic showing probe dissociation strategy with a fluorescently labeled probe designed to hybridize to the 5′ region of the aptamer and dissociate in the presence of cortisol.

FIG. 3B is a heat map showing the optimization of stem #1 length for probe hybridization.

FIG. 3C is a heat map showing the optimization of stem #1 length for a cortisol-induced probe dissociation signal.

FIG. 3D is a diagram of the optimized CSS.3 sequence (5′deletion 2, 3′deletion 1) and fluorescently labeled probe is shown with complementary region highlighted in dark gray.

FIG. 3E is a heatmap that shows cortisol-induced probe dissociation for all possible point mutations of the CSS.3 parent sequence and identifies two regions (loop #2 and loop #3) permissible to mutation. Mutation introduced is shown along the y-axis, and position along the aptamer is shown on the x-axis. FIG. 3E is in gray scale in which darker color represents better binding and lighter shades represent worse binding.

FIG. 4A is a diagram showing regions selected for nucleotide randomization in loop #2 which are circled on the left side, and stem and loop #3 which are circled on the right side, respectively.

FIG. 4B is a dose-response curve showing increase in probe dissociation for the mutant compared to the CSS.3 parent sequence and the scrambled negative control sequence in the presence of cortisol.

FIG. 4C is a dose-response curve showing no probe dissociation for the mutant, CSS.3 parent sequence or the scrambled sequence in the presence of DHEAs.

FIG. 4D is a graph of the specificity of optimized mutant showing strong response to cortisol and 11-deoxycortisol, moderate response to progesterone and no response to other structurally similar targets.

FIG. 4E is a table of the binding characterization on the BLI that demonstrates that the selected mutant has 3-fold better binding affinity K_{D} and selectivity against progesterone compared to the stem-optimized CSS.3_cut3 sequence.

FIG. 5 is a graph showing the chemical shift for the cortisol methyl protons in the aliphatic portion of the NMR spectra.

FIG. 6 is a diagram that shows the structures of the steroid hormones cortisol, DHEAs, progesterone, estradiol, and 11-deoxycortisol, and neurotransmitters dopamine and serotonin.

FIG. 7 is a perspective view of one embodiment of an electrochemical sensor.

FIG. 8 is a schematic drawing that shows a sequence that switches from a structure conformation to a less structure conformation.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity of illustration.

### DETAILED DESCRIPTION OF THE INVENTION

The present invention relates generally to aptamers, biosensors and, more particularly, to aptamer-based biosensors for monitoring cortisol and other hormones. Methods of optimizing aptamers for biosensing applications are also provided.

### Abbreviations

BLI—biolayer Interferometry
BSA—bovine serum albumin
EAB—electrochemical aptamer-based sensor
ISF—interstitial fluid
kDa—kilodalton
MWCO—molecular weight cutoff
NMR—nuclear magnetic resonance
SELEX—systematic evolution of ligands by exponential enrichment
SD—standard deviation
sISF—simulated interstitial fluid
ssDNA—single-stranded DNA
SSA—structure-switching aptamer

Aptamers are nucleic acid ligands capable of binding to molecular targets. Such functional biomolecules may, therefore, be referred to as either aptamers or ligands. The aptamers described herein comprises short single-stranded DNA sequences that have been optimized to bind cortisol and other hormones. The aptamers may be truncated and mutated versions of existing aptamers. Some existing aptamers are described in U.S. Patent application Ser. No. 2019/0136241 A1, Stojanovic, et al. (which describes an invention which was made with U.S. government support), the disclosure of which is incorporated by reference herein. One particular existing aptamer described in the Examples below is known as CSS.3, which is considered to be a three-way junction (3JW) aptamer. However, it is expressly not admitted that the present aptamers are either known or obvious in view of existing aptamers.

The present aptamers are truncated and mutated to improve cortisol binding affinity and, in some cases, are believed to provide structure switching capability in relevant biofluids. The aptamers bind to cortisol and are also expected to bind 11-deoxycortisolc, which is a cortisol metabolite. The aptamers of the present invention may comprise any one of SEQ IDs 1-24 shown in Table 1. In some cases, the aptamers are selected from the group consisting of SEQ IDs 1-24.

TABLE 1
SEQ ID Nos. 1-24

| SEQ ID | |
|---|---|
| No. | Sequence |
| 1 | CTCGGGACGACGCCAGAACGTCAGGAGGATATGGTAACATAGTCGTCC |
| 2 | CTCGGGACGACGCCAGAAAGATATGAGGATAGGGCGACCTAGTCGTCC |
| 3 | CTCGGGACGACGCCAGAAAGATATGAGGATAGGTACGCCTAGTCGTCC |
| 4 | CTCGGGACGACGCCAGAAAGGTATGAGGATAGGCTCGCCTAGTCGTCC |
| 5 | CTCGGGACGACGCCAGAAAGGTATGAGGATAGGGCTCCCTAGTCGTCC |
| 6 | CTCGGGACGACGCCAGAACGATAGGAGGATATGCCTGCATAGTCGTCC |
| 7 | CTCGGGACGACGCCAGAACGATAGGAGGATAGGCTTGCCTAGTCGTCC |
| 8 | CTCGGGACGACGCCAGAACGATAGGAGGATATGCTTGCATAGTCGTCC |
| 9 | CTCGGGACGACGCCAGAACGATAGGAGGATAGGGCTACCTAGTCGTCC |
| 10 | CTCGGGACGACGCCAGAACGATAGGAGGATAGGGCTCCCTAGTCGTCC |
| 11 | CTCGGGACGACGCCAGAACGATAGGAGGATAGGTTTACCTAGTCGTCC |
| 12 | CTCGGGACGACGCCAGAACGTAAGGAGGATAGGCAGACCTAGTCGTCC |
| 13 | CTCGGGACGACGCCAGAACGTAAGGAGGATATGCCTGCATAGTCGTCC |
| 14 | CTCGGGACGACGCCAGAACGTAAGGAGGATAGGCTCGCCTAGTCGTCC |
| 15 | CTCGGGACGACGCCAGAACGTAAGGAGGATATGCTCGCATAGTCGTCC |
| 16 | CTCGGGACGACGCCAGAACGTAAGGAGGATAGGCTTGCCTAGTCGTCC |
| 17 | CTCGGGACGACGCCAGAACGTAAGGAGGATATGCTTGCATAGTCGTCC |
| 18 | CTCGGGACGACGCCAGAACGTAAGGAGGATATGGTTACATAGTCGTCC |
| 19 | CTCGGGACGACGCCAGAACGTAAGGAGGATAGGTTTACCTAGTCGTCC |
| 20 | CTCGGGACGACGCCAGAACGTTAGGAGGATAGGCTTGCCTAGTCGTCC |
| 21 | CTCGGGACGACGCCAGAACGTTAGGAGGATATGCTTGCATAGTCGTCC |
| 22 | CTCGGGACGACGCCAGAACGTTAGGAGGATAGGGCAACCTAGTCGTCC |
| 23 | CTCGGGACGACGCCAGAACGTTAGGAGGATAGGGCTCCCTAGTCGTCC |
| 24 | CTCGGGACGACGCCAGAACGTTAGGAGGATAGGTTTACCTAGTCGTCC |

All of the sequences are synthetic DNA sequences. The sequences may also include any modifications, variants, concatenations, and/or truncations thereto, and in general may include any sequence with substantial or significant homology or sequence identity with the aptamer sequences described herein. The terms “having substantial homology or significant homology or sequence identity”, as used herein, refers to sequences having at least 95% homology or identity to a given sequence, or in the case of a group of sequences, to sequences having at least 95% homology or identity to a sequence selected from the group. In some cases, the sequences may include sequences having at least 50%, 55%, 60%, 65%, 70%, 75%, 80%, 85%, 90%, 95%, or 99% homology or identity to a given sequence or to a sequence selected from the group. In the case of variants, in some cases, 20%, 15%, 10%, 5% or less of the deoxyribonucleotides of the sequence may be changed.

In some embodiments, aptamer-based biosensors are provided. The biosensors can be of any suitable type including, but not limited to: electrochemical (that is, it may be an electrochemical aptamer-based or EAB sensor), colorimetric, or fluorescence based sensors. In some cases, the sequences can be functionalized with a redox reporter (e.g., methylene blue (MB)) and immobilized onto a sensor surface for electrochemical detection of cortisol in biofluids. In some cases, the sequences can be immobilized onto gold sensors using a thiol tag.

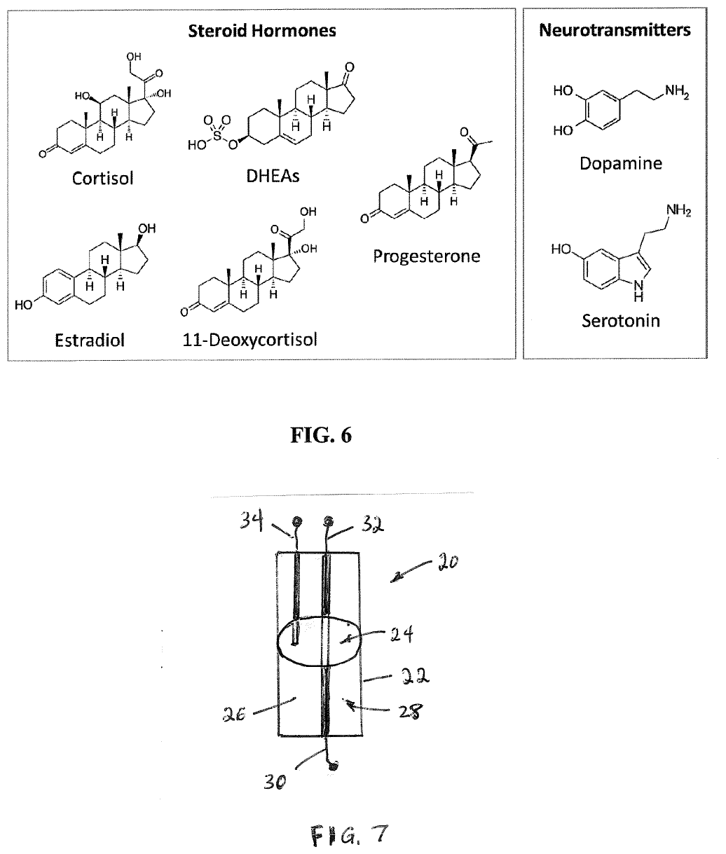

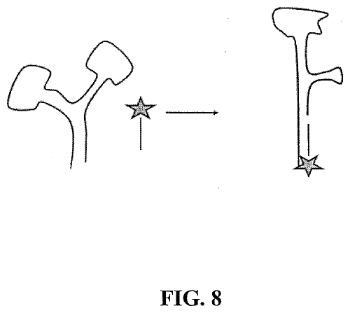

One non-limiting example of an aptamer-based biosensor is shown in FIG. 7. FIG. 7 shows that the biosensor 20 may comprise a glass substrate 22, a microwell 24, a glass coverslip 26, a SU8 resist 28, a working electrode 30, a reference electrode 32, and a counter electrode 34.

Aptamer-based biosensors are also described in U.S. Pat. No. 7,470,516 B2, Stojanovic, et al.; U.S. Pat. No. 11,162,960 B2, Xiao, et al.; U.S. Pat. No. 11,656,235 B2, Xiao, et al.; and U.S. Patent Application Pub. No. US 2023/0213471 A1, Xiao, et al. (which all describe inventions made with U.S. government support), the disclosures of which is incorporated by reference herein. The Stojanovic, et al. patent describes a fluorescence-based sensor. U.S. Pat. No. 11,656,235 B2, Xiao, et al. describes a colorimetric sensor. U.S. Patent Application Pub. No. US 2023/0213471 A1, Xiao, et al. describes methods for fabricating electrochemical aptamer-based (EAB) sensors. The present aptamer-based biosensors, and other aspects of the inventions described herein, may include any of the features described in the Stojanovic, et al. and Xiao, et al. patents and patent application. However, it is expressly not admitted that the present aptamer-based biosensors, or other inventions described herein, are either known or obvious in view of the aptamer-based biosensors described in these patents and patent applications.

In some embodiments, methods of cortisol and other hormone detection are provided. The methods may comprise the steps of contacting a sample with the aptamer-based biosensors described herein, and detecting cortisol or another hormone in the sample. The detection may comprise measuring a signal generated upon binding of the cortisol or other hormone to the aptamer in the aptamer-based biosensor.

In some embodiments, methods of optimizing aptamers for biosensing applications are provided. The following describes the discovery of the improved cortisol binding aptamers and aptamers for binding other target molecules described herein. These aptamers may undergo a conformational change when binding to cortisol or other target molecules. These aptamers do not require the presence of (that is, they are free of) a complementary displacement strand. Additionally, the aptamer sequences described herein show improved binding under physiological conditions (e.g., in simulated and real biofluids and at increased temperatures).

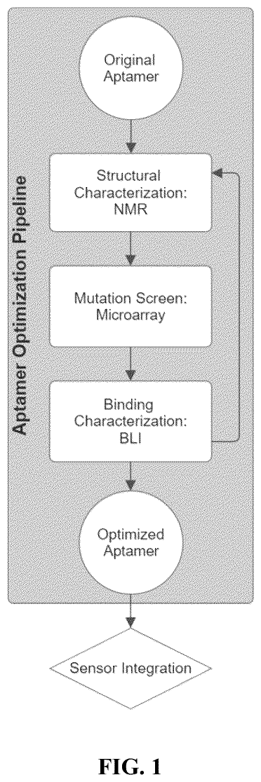

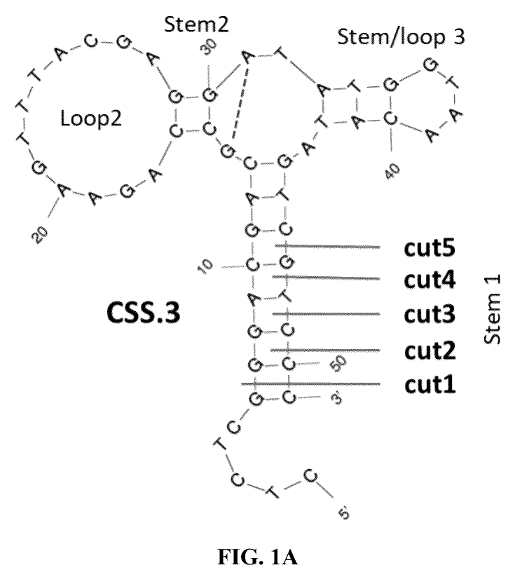

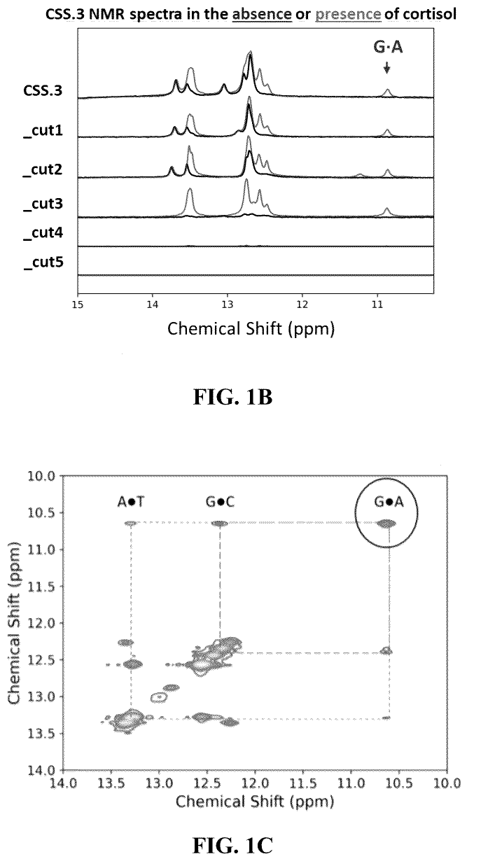

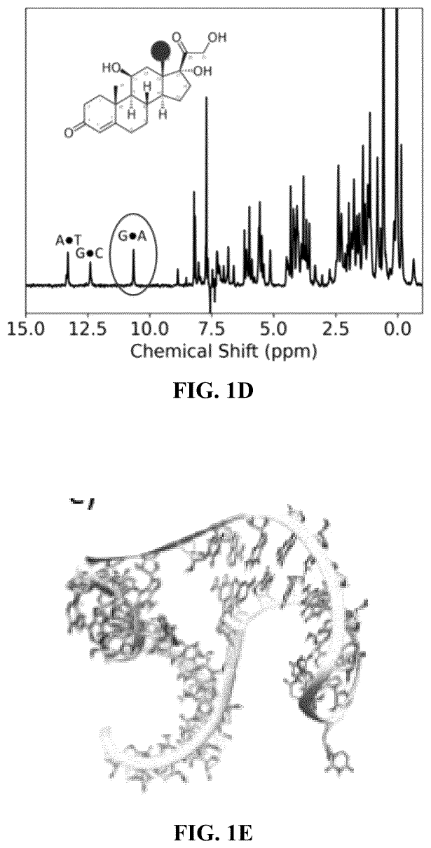

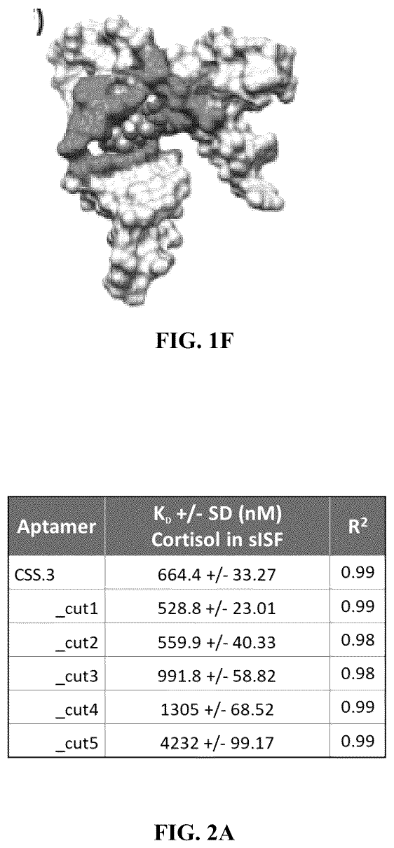

FIG. 1 is an overview of one embodiment of the bio-recognition element (BRE) discovery and optimization pipeline that led to the discovery of improved cortisol binding aptamers described herein. As shown in FIG. 1 there are several steps in this process. These steps may include: a) structural characterization with NMR; b) mutation screening using microarrays (which may involve multiple rounds of microarray screening); and c) binding characterization. These steps may be followed by sensor integration. This process or method may be compatible with a range of sample matrixes (including biofluids), diverse target molecules (such as peptides, proteins, and small molecules), and sensor platforms.

The terms “target” and “small molecule”, as used herein, include any molecule capable of being detected using an aptamer technique. In some embodiments, the small molecule has a molecular weight less than (or less than or equal to) 1,000, 900, 800, 700, 600, 500, 400, 300, or 200 Daltons. The term “small molecule” includes, but is not limited to cortisol and other hormones.

In some embodiments, methods of optimizing aptamers for biosensing applications are provided. In some cases, the aptamers may be conformation-switching. In some cases, a method of optimizing aptamers for biosensing applications may comprise the steps of:
a) structurally characterizing an aptamer at the molecular level using nuclear magnetic resonance (NMR) and computer modeling to identify sequence-specific binding features and conformations, wherein said structural characterization is performed:
   1) in the absence of a target molecule; and
   2) in the presence of a target molecule;
b) performing large-scale mutational analysis using a microarray-based platform to allow for simultaneous assessment of a plurality of aptamer point mutations and rapid screening of combinatorial mutations for improved binding affinity; and
c) characterizing aptamer binding affinity wherein binding affinity is evaluated in at least one of: 1) buffer, 2) biofluid.

These steps are summarized below. Further details on the steps are described in the Test Method section below. These steps can be performed in any suitable order. Typically, the NMR step will occur first to guide the design of the mutants with the microarray experiments. In some cases, one or more steps may be performed simultaneously. For example, binding characterization can be done at any point with parent or mutant sequences. In some cases, one or more steps may be omitted. In some cases, one or more steps may be repeated and/or additional steps may be added.

The first step may comprise a) structurally characterizing an aptamer at the molecular level using nuclear magnetic resonance (NMR) and computer modeling (in silico modeling) to identify sequence-specific binding features and conformations. The use of nuclear magnetic resonance (NMR) involves obtaining the spectra of the DNA aptamer dissolved in a solvent suitable for aptamer folding. The chemical shifts observed in the resulting spectra provide information about the identity and quantity of nucleotide pairing and are used to assess aptamer structure. The aptamer and variations or truncations thereof are analyzed: 1) in the absence of a target molecule; 2) in the presence of a target molecule; and 3) at different temperatures. The formation of new peaks or shifts in existing peaks for an aptamer in the presence of a target molecule indicates structural changes that could be the result of nucleotides pairing, unpairing, and/or interacting with the target molecule. From the NMR spectra, an aptamer with the optimal truncation for maximal structure switching in response to target molecule binding may be selected. In some cases, this may be thought of as a stem and cut work step.

In addition, a 2D Nuclear Overhauser Spectroscopy (NOESY) NMR analysis may be performed on the optimized aptamer with the goal of identifying the target molecule binding site. The spectra in the 2D NOESY NMR analysis may be acquired in physiologically-relevant solutions such as phosphate buffered saline (PBS) or simulated interstitial fluid (sISF).

The structural insights gained from NMR can be used to guide in silico models in the generation of a rigid three-dimensional (3D) structure of the aptamer which is then used as the input for docking studies between the aptamer and the target molecule. Because no reliable tools exist for rigid 3D ssDNA modeling, the initial structure can be generated using RNA modeling software and then converted into DNA before subjecting it to molecular dynamics simulations (see Experimental section below for details).

The second step may comprise performing large-scale mutational analysis to optimize the aptamer sequences using a microarray-based platform to allow for simultaneous assessment of a plurality of aptamer point mutations and rapid screening of combinatorial mutations for improved binding affinity. In some cases, the mutational analysis can be used to allow simultaneous assessment of all possible aptamer point mutations. Mutant libraries are generated and screened on custom microarray chips. The step of high throughput screening may comprise the preparation of microarray platforms containing as many as 1 million DNA sequences per microarray. For cortisol work, the large-scale mutation analysis can typically involve mutation analysis of from about 240 to about 65,000 sequences. The arrays are exposed to multiple concentrations of cortisol as well as structurally similar counter targets to assess binding affinity and specificity. Multiple rounds of microarray screening may be performed. Mutants with the best binding signal and specificity are down-selected for further testing. DNA aptamers may be synthesized and functionalized with relevant moieties (e.g., biotin, thiol, MB (methylene blue)) for testing and sensor integration.

The first and second steps may be performed in the presence of biofluids relevant to continuous sensing application. Such biofluids may comprise at least one of: blood, plasma, urine, tears, saliva, sweat, interstitial fluid (ISF), and simulated interstitial fluid (sISF).

While a robust conformational change is important for sensor integration, it is also important to maintain high binding affinity between the aptamer and target. The third step may comprise characterizing aptamer binding affinity wherein the binding affinity is evaluated in at least one of: 1) buffer, 2) biofluid. The step of binding characterization may comprise the use of one or more of: biolayer interferometry (BLI), isothermal calorimetry (ITC), surface plasmon resonance (SPR), or a plate-based assay such as enzyme linked immunosorbent assay (ELISA) to measure the binding affinity for each of the aptamer variants. In one version of the method, binding affinity and kinetics of selected mutants is determined using biolayer interferometry (BLI). Biotinylated aptamers are immobilized onto streptavidin coated biosensor tips and dipped into wells containing cortisol. Steady state binding affinity (K_{D})) is determined after double reference subtraction.

FIG. 1 shows an arrow connecting the Binding Characterization step back to the Structural Characterization with NMR step. This means that when a promising candidate is identified, steps may be repeated to verify that it goes through a significant conformational change and/or to further improve upon binding characteristics needed for sensor integration. These steps may be repeated two or more times.

There are numerous, non-limiting embodiments of the invention. For example, the method described herein is applicable to aptamers for different targets (such as peptides, proteins, and small molecules other than cortisol).

The method described herein may be used to identify sequences switch from an unstructured (or unfolded) conformation to a structured (or folded) conformation. In another example, as shown in FIG. 8, the method may also be used to identify sequences that switch from a structure conformation (e.g., a folded conformation on the left side of FIG. 8) to a less structure conformation (e.g., less folded conformation on the right side of FIG. 8) upon binding, which may be attractive for certain applications. In this case, NMR is used to confirm the aptamer nucleotides pairing in the absence of the target and characterize the change in pairing patterns upon interacting with the analyte of interest. This may result in a new structure that is more capable of binding the capture probe in a competitive assay, resulting in a signal-on readout in the microarray.

All embodiments, even if they are only described as being “embodiments” of the invention, are intended to be non-limiting (that is, there may be other embodiments in addition to these), unless they are expressly described as limiting the scope of the invention. Any of the embodiments described herein can also be combined with any other embodiments in any manner to form still other embodiments.

The aptamers and aptamer-based biosensors described herein can provide a number of advantages. It should be understood, however, that these advantages need not be required unless they are set forth in the appended claims. They also do not bind cortisol with high enough affinity for sensing in biofluids. The optimized aptamers described herein have improved binding affinity with cortisol, are believed to be structure switching, and are believed to be compatible with continuous EAB sensing. These aptamers do not require the presence of (that is, they are free of) a complementary displacement strand. Additionally, the aptamer sequences described herein show improved binding under physiological conditions (e.g., in simulated and real biofluids and at increased temperatures). The aptamer-based biosensors described herein, therefore, can be used in a variety of electrochemical sensing platforms including microneedle sensing devices. The method described herein is a streamlined approach to SSA optimization in comparison to the typically complex and laborious development process of SSAs. The method described herein is applicable to any other structural scaffold not just aptamers such as CSS.3, which is in the form of a three-way junction (3JW).

### Examples

The examples initially focus on cortisol as a model system. While the examples focus on the structural characterization and optimization of a cortisol binding aptamer, this approach can be broadly applied to optimize SSAs for other relevant targets and support the development of real-time, continuous biomarker sensors.

### Aptamer Structural Characterization and Switching

For this work, a cortisol binding aptamer discovered by Yang, K. A. et al. described in High-Affinity Nucleic-Acid-Based Receptors for Steroids ACS Chem. Biol. 12, 3103-3112 (2017) using a strand displacement SELEX technique is used. The aptamer, named CSS.3, which has a sequence CTC TCG GGA CGA C GC CAG AAG TTT ACG AGG ATA TGG TAA CAT A GTC GTC CC was one of three cortisol aptamers reported by Yang (CSS.1, CSS.2, and CSS.3), but demonstrated the highest affinity for cortisol compared to the other two sequences. Our work confirmed that of the three aptamers, CSS.3 had the best binding affinity of the three in binding buffer with a K_{D} of 249 nM.

FIGS. 1A-F show the structural characterization of CSS.3 aptamer and stem truncations. Structurally, CSS.3 is predicted to form a three-way junction motif consisting of a central loop flanked by three stem regions. The MFold predicted structure is shown in FIG. 1A. Although originally selected using a strand displacement technique, which is common for the identification of structure switching aptamers, through extensive literature research we could not find experimental data demonstrating the aptamer is structure switching, nor structural characterization data.

Using NMR to characterize the aptamer structure, FIG. 1B shows that the CSS.3 aptamer has peaks in the 13-14 and 12-13 ppm range (top line, black trace), corresponding to AT and GC base pairs, respectively. FIG. 1B shows that the addition of cortisol results in the appearance of new peaks, most notably a distinct peak appearing at 10.9 ppm (top line, gray trace). It is believed that this new peak is due to the formation of a non-canonical GA base pair forming in the central loop region adjacent to stem 2 (denoted by the dashed line in FIG. 1A. Because it is only formed in the presence of cortisol, it is believed that the cortisol binding site is partially formed by this GA base pair. This is a notable finding because the presence of a non-canonical base pair forming within the binding pocket cannot be predicted using modeling software alone. CSS.3_cut3 appears to be unstructured in solution but still retains the ability to bind cortisol. Cortisol binding is also confirmed by large chemical shifts for the cortisol methyl protons in the aliphatic portion of the CSS.3 NMR spectra shown in FIG. 5.

Based on this structural characterization work, it is believed that the CSS.3 aptamer is mostly folded in the absence of cortisol, and would be unlikely to undergo a sufficiently large conformational switch for signal generation without the addition of a displacement strand. Recently, a wearable cortisol sweat sensor was developed using the CSS.3 sequence. However, in line with our findings here, it is believed that the aptamer was modified into a pseudoknot structure to assist with conformation-switching for electrochemical signal generation. It is hypothesized that because the aptamer was selected using a strand displacement technique, further optimization would be needed in order to engineer a robust conformational change without the presence of a complementary displacement strand.

To introduce structure switching, the effect of truncating the stem #1 region of the aptamer was systematically examined, as depicted by the cuts shown in FIG. 1A (the sequences of the CSS.3 truncated aptamers are provided in Table 2).

TABLE 2
Sequences of CSS.3 Truncated Aptamers

| | # base | |
|---|---|---|
| | pairs in | |
| | Stem | |
| Aptamer | #1 | Sequence (5′→3′) |
| CSS.3 | 8 | CTCTCGGGACGACGCCAGAAGTTTACGAGGATATGGTAACATAGTCGTCCC |
| _cut1 | 7 | GGACGACGCCAGAAGTTTACGAGGATATGGTAACATAGTCGTCC |
| _cut2 | 6 | GGACGACGCCAGAAGTTTACGAGGATATGGTAACATAGTCGTC |
| _cut3 | 5 | GGACGACGCCAGAAGTTTACGAGGATATGGTAACATAGTCGT |
| _cut4 | 4 | GGACGACGCCAGAAGTTTACGAGGATATGGTAACATAGTCG |
| _cut5 | 3 | GGACGACGCCAGAAGTTTACGAGGATATGGTAACATAGTC |

Based on similar rational design approaches in the literature, it was expected that there would be an increase in structure-switching capability but a loss of binding affinity as the length of the stem region was reduced. However, it was unclear from computationally derived data at what point the optimal balance for switching would occur. To assess structure switching in the truncated aptamers, the aptamer structure was examined in the presence and absence of cortisol using NMR.

FIG. 1B shows the spectra for all CSS.3-truncated aptamers in the absence of cortisol (black traces) and in the presence of cortisol (gray traces). With each sequential aptamer truncation there is a loss of structure, as evidenced by the loss of peaks in the 12-14 ppm region. With the removal of three base pairs (_cut3) the aptamer no longer shows any discernable peaks in this region, which is consistent with the aptamer being unstructured in solution. Notably, once exposed to cortisol, this truncation still retains the ability to bind cortisol, as evidenced by the presence of peaks in the 12-14 ppm region and the GA base pair indicated by the peak at 10.9 ppm. No imino or upfield proton peaks are observed for CSS.3_cut4 or _cut5 in either spectrum, indicating that these sequences remain unstructured even in the presence of cortisol. From the NMR spectra, CSS.3_cut3 is selected as the optimal truncation for maximal structure switching in response to cortisol binding.

In addition, a 2D NOESY NMR analysis was performed on the stem-optimized CSS.3_cut3 aptamer with the goal of identifying the cortisol binding site. The spectra shown in FIG. 1C was acquired in phosphate buffered saline (PBS) (little difference was found between DNA duplexes tested in PBS vs sISF) and at a lower temperature (283 K) to decrease the water exchange rate with a mixing time of 200 milliseconds so that protons separated by longer distances, including those between neighboring imino protons. (The components of sISF are in Table 3 below.)

TABLE 3
Components of Simulated Interstitial Fluid (sISF)

| Reagent | Concentration | |
|---|---|---|
| CaCl₂ | 2.5 | mM |
| Glucose C₆H₁₂O₆ | 5.5 | mM |
| HEPES | 10 | mM |
| KCl | 3.5 | mM |
| MgSO₄ | 0.7 | mM |
| NaCl | 123 | mM |
| NaH₂PO₄ (Sodium phosphate Monobasic) | 1.5 | mM |
| Saccharose/Sucrose C₁₂H₂₂O₁₁ | 7.4 | mM |
| BSA (optional) | 0.3 | mM |

FIG. 1D shows a cross section through the NOESY spectra. These data show numerous exchange peaks that arise both from inter and intramolecular magnetization exchange. Of particular interest are the cross peaks to the imino protons from the GC and AT base pairs, as well as the GA base pair within the central loop. These data confirm that cortisol binds within the central loop of the three-way junction structure and is in close proximity to the GA base pair.

The structural insights gained from NMR were used to model the binding of the CSS.3_cut3 aptamer to cortisol. Because no reliable tools exist for rigid 3D ssDNA modeling, the initial structure was generated using RNA modeling software and then converted into DNA before subjecting it to molecular dynamics simulations (see Experimental section below for details). When selecting structures from the large amount of structures generated, we looked for models where the GA base pair in the central region would be likely to form due to proximity of the two nucleotides. This process generated a rigid 3D structure of CSS.3_cut3 (shown in FIG. 1E, with the GA base pair highlighted in green), which was docked to cortisol. The 3D docked model shows binding occurring within the central loop of the aptamer and with extensive interactions between base pairs in stems 1 and 3, as well as the GA base pair (see FIG. 1F, where the interacting nucleotides are highlighted in magenta).

### Sequence Optimization for Improved Affinity

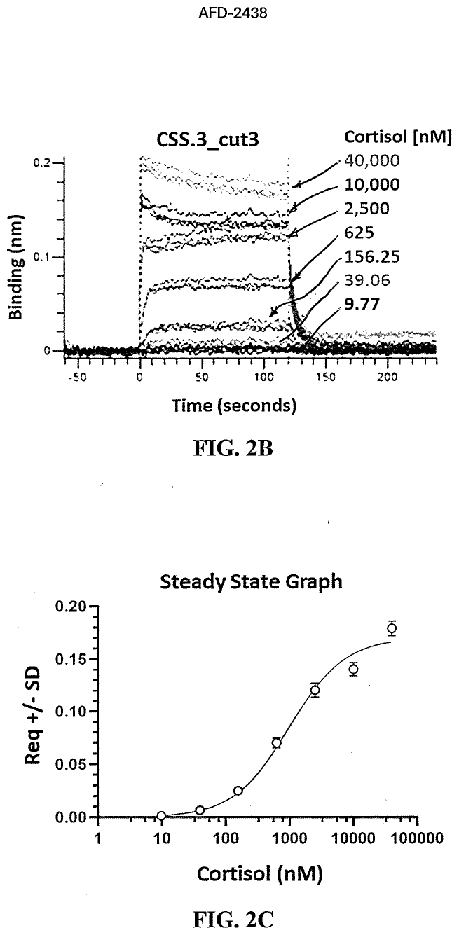

While a robust conformational change is important for sensor integration, it is also important to maintain high binding affinity between the aptamer and target. Biolayer Interferometry (BLI) was used to measure the binding affinity for each of the CSS.3 aptamer variants in sISF. FIGS. 2A-C show binding for all CSS.3 aptamer truncations measured using BLI. The measured binding affinities for each of the aptamer truncations are shown in FIG. 2A. CSS.3_cut3 shows some loss of cortisol binding affinity with a K_{D} of 991.8 nM compared to 664.4 nM for the full-length aptamer. CSS.3_cut and _cut2 show slight improvement in binding affinity, likely due to easier folding with the removal of non-essential nucleotides from the 3′ end. Removal of more than three base pairs from the stem #1 region results in more significant loss of binding affinity as the K_{D} value for cut 5 was found to be 4232 nM. These findings are consistent with the NMR findings in that removal of more than three bases from the stem region results in destabilization of the aptamer structure, such that cortisol binding affinity is negatively affected. The overlaid traces in FIG. 2B shows real-time baseline, association, and dissociation to cortisol and FIG. 2C shows the steady state curve fitting results for CSS.3_cut3.

The results from binding affinity determination highlight another important finding. When tested in sISF, a more biologically relevant and complex buffer, the CSS.3 aptamer undergoes an almost 3-fold loss of affinity (the K_{D} for CSS.3 dropped from 249 nM in tris binding buffer to 664.4 nM in sISF (FIG. 2A). Given the combined deleterious effects of both stem truncation and biofluid composition on binding affinity, we sought to further improve aptamer performance for sensing applications.

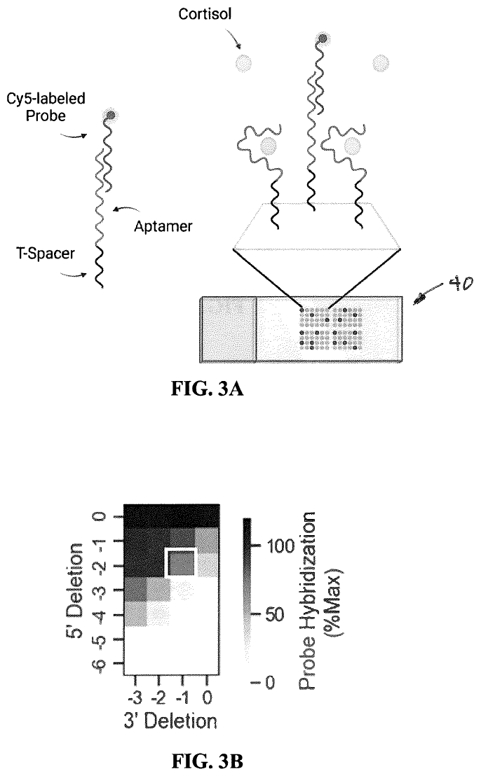

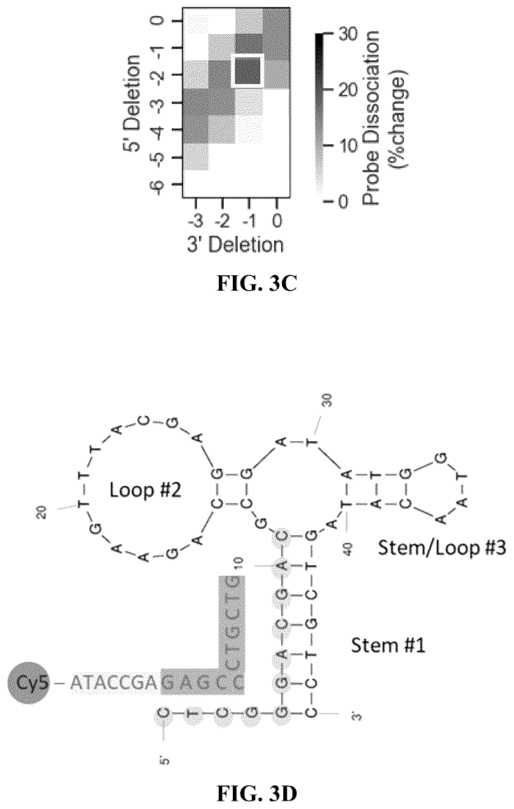

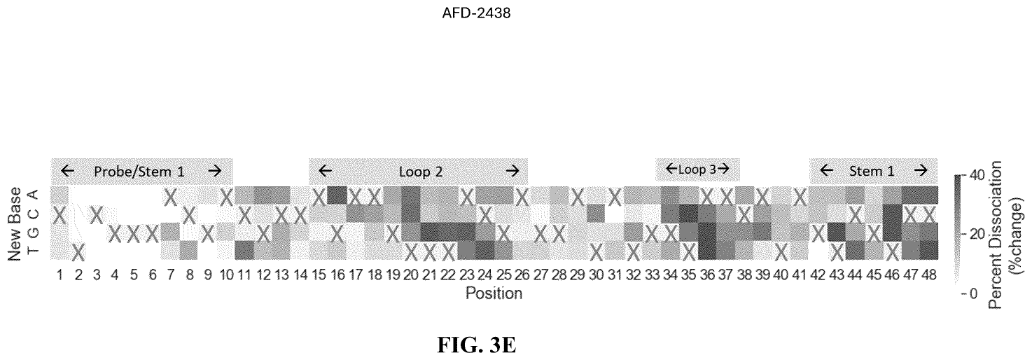

To do this, a large-scale sequence maturation and screening approach was carried out using microarrays, with the goal of identifying better binders in sISF. FIGS. 3A-E show microarray-based target-induced probe dissociation screening. Because cortisol is a small molecule and therefore has limited number of epitopes available for binding, a strand-displacement approach was used for microarray screening as shown schematically in FIG. 3A. FIG. 3A shows a microarray 40 having a plurality of small circles which represent DNA sequences, each of which contains a T-spacer and an aptamer. Like strand-displacement SELEX, a displacement probe was used with a fluorescent label (the Cy5-labeled probe) as an indirect measure of cortisol binding. The T-spacer is a T nucleotide that spaces that aptamer away from the surface of the glass of the microarray. In the absence of cortisol, the probe hybridizes to complementary bases on the 5′ end of the aptamer and forms a duplex. Upon binding to cortisol, the aptamer undergoes a conformational change that opens and displaces the probe, and folds around the cortisol molecule. Unbound probe is then removed during the washing step. The amount of probe dissociation in the presence of cortisol can be calculated relative to a buffer control and is used to estimate binding.

For this approach to be successful, it was important to first optimize the kinetics of probe hybridization. If probe hybridization is too strong, it can prevent the transition of the aptamer into its binding conformation. Conversely, if probe hybridization is too weak, it is likely to dissociate even in the absence of target. To achieve a measurable binding signal for the CSS.3 aptamer, we first studied the effect of 5′ and 3′ truncations on probe hybridization and cortisol-induced probe dissociation (shown in the heat maps in FIGS. 3B and 3C). The full-length CSS.3 parent sequence is represented by the square in the upper right corner of the heatmaps (5′deletion0, 3′deletion0), and combinations of both 5′ and 3′ truncations can be mapped by moving down and left, respectively, across the heatmap.

Regarding probe hybridization (as shown in FIG. 3B), it was found that hybridization weakened as nucleotides were deleted from the 5′ region of the aptamer. This was expected, since the 5′ region is complementary to the probe, and removal of nucleotides from that region would be expected to weaken probe hybridization. Conversely, as nucleotides were deleted from the 3′ region of the aptamer, probe hybridization strengthened because aptamer folding became weaker with fewer base pairs present in stem #1, therefore making it easier for the probe to hybridize.

Regarding cortisol-induced dissociation (as shown in FIG. 3C), an inverse relationship was found to that of probe hybridization. As bases were removed from the 5′ region of the aptamer, cortisol binding increased. Again, this result was expected because probe hybridization would be weakened and easier to displace upon binding. Similarly, cortisol-induced probe dissociation decreased with the removal of bases from the 3′ region. This was believed to be due to the weakening of stem #1, therefore making it harder for the aptamer to fold into its binding conformation and displace the probe. The sequences at the bottom right corner of the heatmap showed no cortisol-induced probe dissociation due to the lack of initial probe hybridization.

The sequences with measurable cortisol binding can be visualized as a diagonal band across the heatmap in FIG. 3C. For microarray screening, the sequence shown in FIG. 3D (5′del2, 3′del1; in the boxes in FIGS. 3B and 3C) was chosen as the optimized parent sequence for microarray screening. Although probe hybridization was slightly lower with this truncation than for the full-length CSS.3 aptamer, it showed high sensitivity to cortisol.

For sequence optimization, we first focused on identifying regions of the aptamer amenable to mutation. It was anticipated that residues close to the binding pocket would have the highest impact on binding affinity. Thus, it was unclear which positions could be mutated without disrupting cortisol binding. Using the microarray, the cortisol responses for all possible single point mutants of the CSS.3 parent aptamer were looked at (as shown in FIG. 3E). Probe dissociation in the heatmap is in gray scale with the darker shades of gray representing a higher percentage disassociation (% change of CSS.3 parent=29%). The data show that most positions were not able to be mutated without negatively affecting cortisol binding. This included mutation of positions 12 and 29; the nucleotides involved in the GA base pair identified from NMR structural characterization. The only regions permissive to mutation were nucleotides 16, 20-24 and 34-37, which correspond to the outer part of loop #2 and loop #3.

While the microarray point mutation findings were largely consistent with the binding pocket identified from NMR, they also highlight the importance of residues not otherwise identified. For example, single point mutations in regions of loop #2 (e.g., positions 17-19) also resulted in loss of binding. These residues were not highlighted as binding sites by either NMR nor structural modeling and would not be expected to be important from a rational design approach, since unpaired bases in loop #2 would be least likely to be involved in stabilizing aptamer binding conformation or interacting directly with cortisol. These findings further highlight the importance of large-scale mutational analysis in sequence optimization approaches. While these examples focus on the optimization of a cortisol binding aptamer, we have also had success in applying this method to aptamers for other targets shown in FIG. 6, such as other steroid hormones like DHEAs, estradiol, 11-deoxycortisol, and progesterone, as well as neurotransmitters such as dopamine and serotonin.

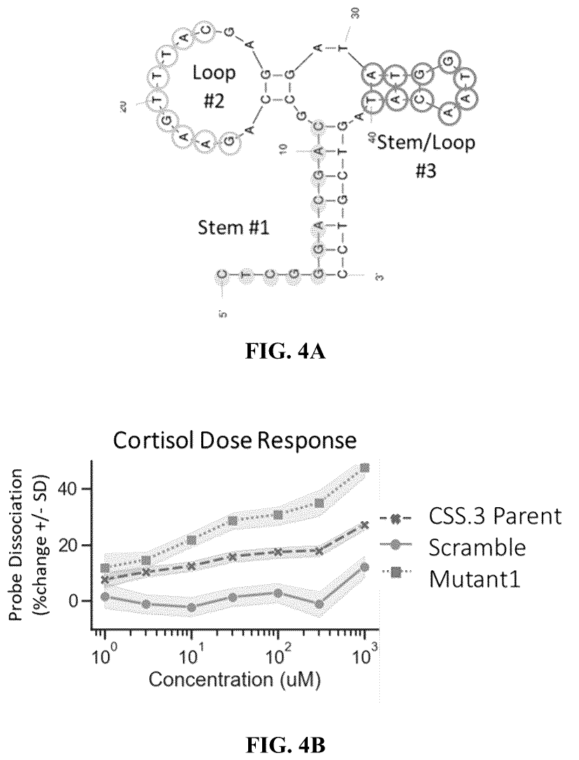

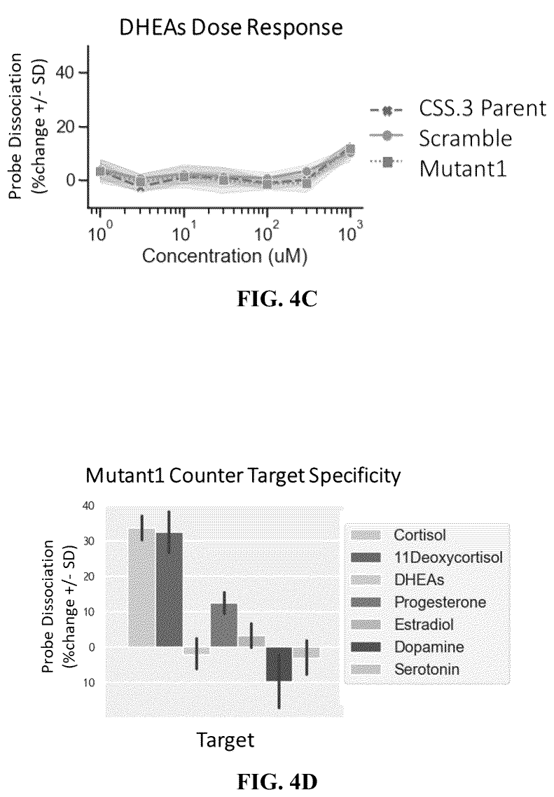

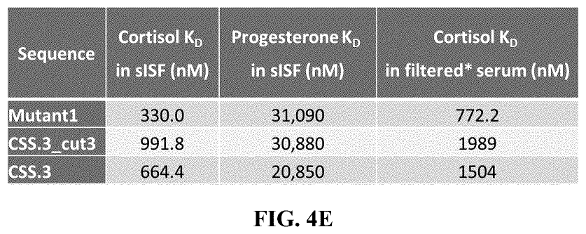

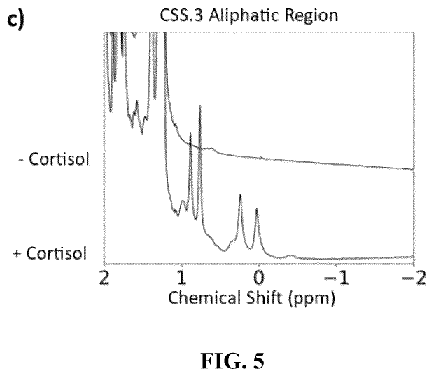

Next, randomization of the regions identified as permissive to mutation was performed. FIGS. 4A-D show the sequence optimization of the CSS.3 aptamer for improved binding affinity. In the first round of sequence optimization, bases in the outer region of loop #2 or stem and loop #3, circled on the left side and the right side, respectively, in FIG. 4A were mutated. Combinations of 2, 3, 4 or 9 nucleotide substitutions were permitted per mutant. This process generated a library of 60,000 sequences that were screened on the microarray for binding to 300 uM cortisol. A total of 24 mutants were identified with similar or better response to cortisol than the CSS.3 parent sequence.

The functional mutations identified in loop #2 and stem and loop #3 were then combined and a second round of sequence optimization was performed. A total of 2,000 additional mutants were generated and screened on the microarray against cortisol (0-1 mM, half log dilutions) and DHEAs. From this second round of screening, mutants with improved sensitivity to cortisol while still maintaining binding specificity against DHEAs were identified. FIGS. 4B and 4C show the dose response curves for a selected mutant (Mutant1) along with the CSS.3 parent and a scramble sequence to demonstrate positive and negative dose-responses for cortisol. None of these sequences showed measurable probe dissociation in the presence of DHEAs.

Additionally, we were able to assess binding specificity for all sequences on the array against a panel of structurally similar counter targets (including 300 uM DHEAs, progesterone, serotonin, dopamine, estradiol, and 11-deoxycortisol; the structures of which are shown in FIG. 6). It was found that all cortisol binding sequences (including the original CSS.3 parent sequence) showed a strong response to both cortisol and 11-deoxycortisol (an immediate metabolite of cortisol with a very short half-life), a weak response to progesterone, and little to no binding to the remainder of molecules tested, including DHEAs, estradiol, dopamine, and serotonin. Specificity data for Mutant 1 is shown in FIG. 4D. The bars in FIG. 4D from left to right coincide with the targets listed from top to bottom. The 12 mutants showing the strongest cortisol binding affinity were down-selected for further characterization using BLI. The results for Mutant I were a roughly 3-fold improvement in binding affinity for cortisol compared to the CSS.3 stem-optimized variant (K_{D} in sISF was 330 nM vs 991.8 nM for CSS.3_cut3; FIG. 4E). This mutant also showed roughly 100-fold selectivity for cortisol over progesterone, which was increased compared to both the full-length CSS.3 aptamer and stem-optimized CSS.3_cut3 (˜30-fold each).

This also shows how the mutant performs in a real biofluid to highlight the translatability of these findings to real-world sensing environments. Because of the difficulties in collecting sufficient volumes of ISF, we used filtered human serum (treated with a 30 kDa molecular weight cutoff (MWCO) filter). Again, the selected mutant maintained roughly 3-fold better binding affinity than the stem-optimized CSS.3_cut3 (FIG. 4E).

### CONCLUSIONS

The example demonstrates an approach to aptamer optimization that uses structural characterization data at the molecular level (NMR) to identify unique sequence motifs critical to aptamer structure and binding. This information is used to guide rational design of an aptamer truncation with robust conformational switching capability in the presence of target. Additionally, use of the microarray platform provided the ability to rapidly study the effect on binding of every possible point mutation in the aptamer sequence, identify regions permissive to mutation, and screen combinations of mutations resulting in the identification of a cortisol-selective mutant with 3-fold improvement in cortisol binding affinity in just two rounds of microarray screening. All screening was performed in a biologically relevant buffer (sISF) and showed direct translation to a human biofluid (filtered human serum). While this example focused on optimization of a cortisol binding aptamer, the same approach is applicable to aptamers for different targets. It is believed that the approach is broadly applicable for the development of SSAs and that this capability will accelerate aptamer sequence optimization to support the development of rapid, continuous, and wearable biosensing applications.

### Test Methods

### Materials

Oligonucleotide sequences were synthesized and high-performance liquid chromatography (HPLC) purified by Integrated DNA Technologies (Coralville, Iowa). All oligonucleotides were dissolved in nuclease-free water at 100 uM and stored at −20° C. Unless noted otherwise, all chemicals and reagents were purchased from Sigma-Aldrich (St. Louis, MO). Target compounds were solubilized in dimethyl sulfoxide (DMSO) (Sigma Aldrich, St. Louis, MO) at a stock concentration of 10 mM. Experiments were performed in phosphate buffered saline (PBS), binding buffer (50 mM Tris-HCl, 300 mM NaCl, 5 mM MgCl, 30 mM KCl, pH 7.4), simulated ISF or pooled human serum (Innovative Research, Novi, MI).

### Nuclear Magnetic Resonance (NMR)

NMR spectra for the aptamer and aptamer-cortisol complexes were obtained at 400, 600 and 800 MHz on Bruker and Tecmag NMR spectrometers. The samples were prepared in sISF containing 10% D2O for NMR locking. The imino proton spectra were observed using the excitation sculpting pulse sequence with a 2 ms water inversion pulse. A concentrated cortisol stock solution was prepared in DMSO-d6 and 5 uL was added to the NMR sample to give the 1:1 complex. The concentration of DNA aptamer was 1.0 mM. Experiments were performed at 25° C. The 2D Nuclear Overhauser Spectroscopy (NOESY) spectra were measured in PBS containing 10% D20 at 800 MHz and 283K with a 200 ms mixing time.

### In-Silico Modeling

The secondary structure and folding energy of the cortisol-binding aptamers were calculated using a UNAFold web server (reference 26) with inputs of 300 mM NaCl, 5 mM Mg2+ and 25° C. for consistency with initial selection buffer conditions. The 3D structures of ssDNA aptamers were generated in two steps. Initially, thousands of tertiary structure models for the equivalent ssRNA sequence were produced using the RNA de novo protocol through Fragment Assembly of RNA with Full Atom Refinement (FARFAR) from the Rosetta package (reference 27). Five models with the lowest energy and with the presence of a non-canonical GA base pair (see NMR results) were selected for the second step to evaluate their stability using Molecular Dynamics simulations. The selected ssRNA structures were converted into ssDNA models by transforming the uracil residues to thymine and replacing the ribose sugar backbone with deoxyribose using the LEaP program of the Amber20 suite of biomolecular simulation programs (reference 28). The obtained DNA molecules were solvated with TIP3P water in a rectangular box with periodic boundary conditions. Na+ and Cl− ions were added to keep the neutrality of the system and the experimental salt concentration. Initially, system was equilibrated at ambient conditions with fixed backbones for 300 ps using the sander program of the Amber20 package. Secondly, the whole system was equilibrated for 300 ps and, finally, the production run was performed for 2 ns. During the production run 100 conformations were saved for stability analysis. The stability of ssDNA structures was determined based on the variability of configurational energy and GA base pair distance. Five conformations of the most stable structure separated by 200 ps of simulations were selected for modeling of cortisol binding. The docking of cortisol to the DNA aptamer was performed using the PatchDock web server developed based on shape complementary principles (reference 29). Both cortisol and aptamer were considered as rigid, and a global search of the rotational and translational space was performed without any constraints on the locations of the binding site. Two cortisol-aptamer complexes with the highest scoring were selected for each of the DNA conformations and analyzed to identify the cortisol-binding site. The visualization of molecules was performed using the UCSF Chimera package (reference 30).

### Bio-Layer Interferometry (BLI)

BLI was used to determine equilibrium dissociation constants (K_{D})) for each aptamer to target molecule. Experiments were performed in binding buffer or sISF at 30° C. Aptamer sequences were synthesized with a 5′ biotin tag (Integrated DNA Technologies, Coralville, Iowa) and immobilized onto SuperStreptavidin biosensor tips (Sartorious, Gottingen, Germany) for binding affinity characterization on the Octet Red96e (Sartorious, Gottingen, Germany). Briefly, aptamer-loaded tips were baselined in the experimental buffer for 30 seconds before binding. An additional 180 second pre-equilibration step was added following aptamer loading for experiments run in sISF to allow for a stable signal prior to baseline. Association and dissociation steps were both 120 seconds long (binding buffer or sISF). Data were double reference subtracted using a buffer only control well and scrambled aptamer sequence. Due to the rapid kinetics of small molecule binding, dissociation constants (K_{D}) were calculated using steady state curve fitting from the average of three replicate runs (one-site specific binding; GraphPad Prism v9.3).

### Microarray

Custom DNA microarrays were synthesized by Agilent Technologies (Santa Clara, CA). Sequences were printed onto the glass surface starting at the 3′ end and linked by a variable length T spacer such that all sequences were 60 nucleotides in length. A minimum of 3 replicates were printed for each sequence. Control sequences were also included for array validation and grid fitting. Prior to screening, microarray slides were rehydrated in a water bath (42° C.) for 30 minutes. Using an Agilent hybridization chamber (including base, cover and clamp assembly) and backing slide, sub arrays were co-incubated for 1 hour with fluorescently-labeled capture probe (100 μM Probe-Cy5; 5′-GTCGTCCCGAGAGCCATA-Cy5-3′) and cortisol (0-300 μM). Counter targets were screened at equimolar concentrations of 300 μM, unless otherwise indicated. All experiments were performed in sISF+BSA. A control array (100 μM Probe-Cy5 only) was included on each slide and used to establish baseline fluorescence for data analysis. After hybridization, slides were washed by dipping for 10 seconds in binding buffer, then PBS. To remove any residual buffer salts before imaging, slides were quickly dipped in nuclease-free water and dried under a stream of nitrogen. Slides were imaged on a fluorescent scanner (Agilent SureScan Microarray Scanner) at a wavelength of 635 nm (red channel) with 3 um resolution and 20-bit dynamic range. Median background-subtracted spot intensities were extracted from each spot using Mapix Analysis software (v1.8, Innopsys) and normalized to compare probe hybridization across all subarrays (eq. 1).

$$Probe Hybridization ( \% Max ) = \frac{( x - Min )}{( Max - Min )} \qquad ( 1 )$$

where x represents the raw fluorescence intensity for a particular spot, and Min and Max represent mean fluorescence intensity for positive and negative probe hybridization control spots, respectively. Target-induced probe dissociation was then calculated as % Change (eq. 2) relative to a blank (probe only) control array.

Probe Dissociation (% Change)=% Max_{blank}−% Max_{target} (2)

Data in graphs represent mean+/−standard deviation (SD) for all replicates of a given sequence.

### REFERENCES

1. Feagin, T. A., Maganzini, N. & Soh, H. T. Strategies for Creating Structure-Switching Aptamers. ACS Sensors 3, 1611-1615 (2018).
2. Wilson, B. D., Hariri, A. A., Thompson, I. A. P., Eisenstein, M. & Soh, H. T. Independent control of the thermodynamic and kinetic properties of aptamer switches. Nat. Commun. 2019 101 10, 1-9 (2019).
3. Nutiu, R. & Li, Y. Structure-switching signaling aptamers. J. Am. Chem. Soc. 125, 4771-4778 (2003).
4. Ma, X. et al. Development of structure-switching aptamers for kanamycin detection based on fluorescence resonance energy transfer. Front. Chem. 7, 29 (2019).
5. Tang, Z. et al. Aptamer switch probe based on intramolecular displacement. J. Am. Chem. Soc. 130, 11268-11269 (2008).
6. Chen, J., Fang, Z., Liu, J. & Zeng, L. A simple and rapid biosensor for ochratoxin A based on a structure-switching signaling aptamer. Food Control 25, 555-560 (2012).
7. Morse, D. P. Direct selection of RNA beacon aptamers. Biochem. Biophys. Res. Commun. 359, 94-101 (2007).
8. Schoukroun-Barnes, L. R. et al. Reagentless, Structure-Switching, Electrochemical Aptamer-Based Sensors. Annu Rev Anal Chem (Palo Alto Calif). 9, 163-181 (2016).
9. Yoshikawa, A. M. et al. A massively parallel screening platform for converting aptamers into molecular switches. Nat. Commun. 14, (2023).
10. Lam, S. Y., Lau, H. L. & Kwok, C. K. Capture-SELEX: Selection Strategy, Aptamer Identification, and Biosensing Application. Biosensors 12, (2022).
11. Lyu, C., Khan, I. M. & Wang, Z. Capture-SELEX for aptamer selection: A short review. Talanta 229, 122274 (2021).
12. Yang, K. A., Pei, R. & Stojanovic, M. N. In vitro selection and amplification protocols for isolation of aptameric sensors for small molecules. Methods 106, 58-65 (2016).
13. Parlak, O. Portable and wearable real-time stress monitoring: A critical review. Sensors and Actuators Reports 3, 100036 (2021).
14. Wang, X. & Walt, D. R. Simultaneous detection of small molecules, proteins and microRNAs using single molecule arrays. Chem. Sci. 11, 7896-7903 (2020).
15. Jones, C. & Gwenin, C. Cortisol level dysregulation and its prevalence—Is it nature's alarm clock? Physiol. Rep. 8, (2021).
16. Torrente-Rodríguez, R. M. et al. Investigation of cortisol dynamics in human sweat using a graphene-based wireless mHealth system. Matter 2, 921-937 (2020).
17. Venugopal, M., Arya, S. K., Chornokur, G. & Bhansali, S. A Realtime and Continuous Assessment of Cortisol in ISF Using Electrochemical Impedance Spectroscopy. Sens. Actuators. A. Phys. 172, 154 (2011).
18. Laudat, M. H. et al. Salivary cortisol measurement: a practical approach to assess pituitary-adrenal function. J. Clin. Endocrinol. Metab. 66, 343-348 (1988).
19. El-Farhan, N., Rees, D. A. & Evans, C. Measuring cortisol in serum, urine and saliva—are our assays good enough? Ann. Clin. Biochem. 54, 308-322 (2017).
20. Gebhart, S. et al. Glucose sensing in transdermal body fluid collected under continuous vacuum pressure via micropores in the stratum corneum. Diabetes Technol. Ther. 5, 159-166 (2003).
21. Wu, Y. et al. Microneedle Aptamer-Based Sensors for Continuous, Real-Time Therapeutic Drug Monitoring. Anal. Chem. (2022).
22. Lin, S. et al. Wearable microneedle-based electrochemical aptamer biosensing for precision dosing of drugs with narrow therapeutic windows. Sci. Adv. 8, 4539 (2022).
23. Yi, K. et al. Aptamer-decorated porous microneedles arrays for extraction and detection of skin interstitial fluid biomarkers. Biosens. Bioelectron. 190, 113404 (2021).
24. Hwang, T. L. & Shaka, A. J. Water Suppression That Works. Excitation Sculpting Using Arbitrary Wave-Forms and Pulsed-Field Gradients. J. Magn. Reson. Ser. A 112, 275-279 (1995).
25. Cavanagh, J., Fairbrother, W. J., Palmer, A. G., Rance, M. & Skelton, N. J. Protein NMR Spectroscopy: Principles and Practice. Protein NMR Spectrosc. Princ. Pract. 1-915 (2006).
26. UNAFold Web server at <http://www.unafold.org/mfold/applications/dna-folding-form.php>
27. Cheng, C. Y., Chou, F. C. & Das, R. Modeling Complex RNA Tertiary Folds with Rosetta. Methods Enzymol. 553, 35-64 (2015).
28. Cheatham, T. E. & Case, D. A. Twenty-five years of nucleic acid simulations. Biopolymers 99, 969-977 (2013).
29. Schneidman-Duhovny, D., Inbar, Y., Nussinov, R. & Wolfson, H. J. PatchDock and SymmDock: servers for rigid and symmetric docking. Nucleic Acids Res. 33, W363 (2005).
30. Pettersen, E. F. et al. UCSF Chimera—a visualization system for exploratory research and analysis. J. Comput. Chem. 25, 1605-1612 (2004).
31. Yang, K. A. et al. High-Affinity Nucleic-Acid-Based Receptors for Steroids. ACS Chem. Biol. 12, 3103-3112 (2017).
32. Niu, C., Ding, Y., Zhang, C. & Liu, J. Comparing two cortisol aptamers for label-free fluorescent and colorimetric biosensors+. Cite this Sens. Diagn 1, 541 (2022).
33. Churcher, Z. R., Johnson, P. E. & Johnson, P. NMR for non-experts; a practical guide for applying NMR methods in studies of aptamer-ligand interactions. 4, 3-9 (2020).
34. Aptamers, | & Sakamoto, T. MINI-REVIEW NMR study of aptamers. Aptamers 1, 13-18 (2017).
35. Singh, N. K., Chung, S., Chang, A. Y., Wang, J. & Hall, D. A. A non-invasive wearable stress patch for real-time cortisol monitoring using a pseudoknot-assisted aptamer. Biosens. Bioelectron. 227, 115097 (2023).
36. Catherine, A. T., Shishido, S. N., Robbins-Welty, G. A. & Diegelman-Parente, A. Rational design of a structure-switching DNA aptamer for potassium ions. FEBS Open Bio 4, 788-795 (2014).
37. Schoukroun-Barnes, L. R. & White, R. J. Rationally Designing Aptamer Sequences with Reduced Affinity for Controlled Sensor Performance. Sensors 2015, Vol. 15, Pages 7754-7767 15, 7754-7767 (2015).
38. Neves, M. A. D. et al. Optimizing Stem Length to Improve Ligand Selectivity in a Structure-Switching Cocaine-Binding Aptamer. ACS Sensors 2, 1539-1545 (2017).
39. Kaur, H. & Yung, L. Y. L. Probing High Affinity Sequences of DNA Aptamer against VEGF165. PLOS One 7, 31196 (2012).
40. Heilkenbrinker, A. et al. Identification of the target binding site of ethanolamine-binding aptamers and its exploitation for ethanolamine detection. Anal. Chem. 87, 677-685 (2015).
41. Feagin, T. A., Maganzini, N. & Soh, H. T. Strategies for Creating Structure-Switching Aptamers. ACS sensors 3, 1611-1615 (2018).
42. Wan, L., Yoshikawa, A., Eisenstein, M. & Soh, H. T. High-Throughput Strategy for Enhancing Aptamer Performance across Different Environmental Conditions. ACS Sensors (2023).

As used herein, an element or step recited in the singular and preceded with the word “a” or “an” should be understood as including the plural of such elements or steps, unless the plural of such elements or steps is specifically excluded.

The appendices to the provisional application and the disclosure of all patents, patent applications, and publications mentioned throughout this description, including in the Background of the Invention section are hereby incorporated by reference herein. It is expressly not admitted, however, that any of the documents incorporated by reference herein teach or disclose the present invention.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. An aptamer having at least 95% homology or identity to a sequence selected from the group consisting of SEQ IDs 1-24.

2. The aptamer of claim 1 which is cortisol binding.

3. The aptamer of claim 2 which is a structure-switching aptamer (SSA) in the presence of cortisol.

4. The aptamer of claim 2 which changes from an unfolded to a folded conformation in the presence of cortisol.

5. An aptamer-based sensor comprising the aptamer of claim 1.

6. The aptamer-based sensor of claim 5 comprising a glucocorticoid biosensor.

7. The glucocorticoid biosensor of claim 6 which comprises one of the following types of biosensors: electrochemical, colorimetric, and fluorescence-based sensors.

8. A method of using the aptamer of claim 1 as a sensing molecule.

9. A method for cortisol detection comprising employing the aptamer according to claim 1 as a cortisol sensing molecule.

10. A method for detecting cortisol in a sample comprising the steps of:
contacting the sample with the aptamer-based sensor of claim 5;
detecting cortisol in the sample, the detection comprising measuring a signal generated upon binding of cortisol with the aptamer-based sensor.

11. The method of claim 10 wherein the sample is a biological sample or an environmental sample.

12. The method of claim 11 wherein the sample is a biological sample selected from blood, plasma, urine, tears, saliva, sweat, interstitial fluid (ISF), and simulated interstitial fluid (sISF).

13. The method of claim 10 further comprising determining the concentration of cortisol in the sample.

14. A method of optimizing conformation-switching aptamers for biosensing applications, said method comprising the steps of:
a) structurally characterizing an aptamer at the molecular level using nuclear magnetic resonance (NMR) and computer modeling to identify sequence-specific binding features and conformations, wherein said structural characterization is performed:
1) in the absence of a target molecule; and
2) in the presence of a target molecule;
b) performing large-scale mutational analysis using a microarray-based platform to allow for simultaneous assessment of a plurality of aptamer point mutations and rapid screening of combinatorial mutations for improved binding affinity; and
c) characterizing aptamer binding affinity wherein said binding affinity is evaluated in at least one of: 1) buffer, 2) biofluid.

15. The method of claim 14 wherein the steps a) and b) are performed in the presence of at least one of: blood, plasma, urine, tears, saliva, sweat, interstitial fluid (ISF), and simulated interstitial fluid (sISF).

16. The method of claim 14 wherein in step b) microarray chips are exposed to multiple concentrations of cortisol as well as structurally similar counter targets to assess specificity.

17. The method of claim 16 wherein the structurally similar counter targets comprise at least one of: DHEAs, progesterone, serotonin, dopamine, estradiol, and 11-deoxycortisol.

18. The method of claim 14 wherein characterizing aptamer binding affinity in step c) comprises the use of one or more of: Biolayer Interferometry (BLI), isothermal calorimetry (ITC), or surface plasmon resonance (SPR) to measure the binding affinity for each of the aptamer variants.

19. The method of claim 14 wherein the method is used to identify an aptamer which changes from an unfolded to a folded conformation in the presence of a target.

20. The method of claim 14 wherein the method is used to identify an aptamer which changes from a structure conformation to a less structure conformation in the presence of a target.
